(19) United States
(12) Patent Application Publication
(10) Pub. No.: US 2026/0265669 A1
(43) Pub. Date: Sep. 10, 2026

(54) DETECTION DEVICE AND METHOD FOR MEASURING ANAEROBIC AMMONIUM OXIDATION ACTIVITY

(71) Applicant: CHINA THREE GORGES CORPORATION, Wuhan, Hubei (CN)

(72) Inventors: Yasong CHEN, Wuhan (CN); Xinyu WAN, Wuhan (CN); Dianchang WANG, Wuhan (CN); Guangrong CAO, Wuhan (CN); Chong LI, Wuhan (CN); Hailei MOU, Wuhan (CN); Fangyuan JING, Wuhan (CN); Wanlin LV, Wuhan (CN); Mengmeng LIU, Wuhan (CN)

(73) Assignee: CHINA THREE GORGES CORPORATION, Wuhan, Hubei (CN)

(21) Appl. No.: 19/167,309

(22) PCT Filed: Dec. 19, 2023
(86) PCT No.: PCT/CN2023/140028

§ 371 Date: Sep. 19, 2025

(30) Foreign Application Priority Data
Jul. 19, 2023 (CN) 202310899960.5

(51) Int. Cl. C12M 1/34; C12M 1/00; C12M 1/107; C12M 1/36

(52) U.S. Cl. CPC C12M 41/38; C12M 21/04; C12M 23/38; C12M 23/58; C12M 29/20; C12M 41/12; C12M 41/26; C12M 41/44; C12M 41/48

(57) ABSTRACT

A detection device and method for measuring anaerobic ammonium oxidation activity. The detection device comprises a reactor and a detector; the reactor comprises a first reaction chamber and a second reaction chamber; the first reaction chamber is used for an anaerobic ammonium oxidation reaction and a denitrification reaction of sludge to be tested; the second reaction chamber is used for a denitrification reaction of said sludge; and the detector is used for controlling and recording the flow rates of chemicals entering the first reaction chamber and the second reaction chamber, ensuring that the pH values of the first reaction chamber and the second reaction chamber are constant and the same.

The present application claims priority to Chinese patent application No. 202310899960.5 filed on Jul. 19, 2023 to the China Patent Office, and entitled “DETECTION DEVICE AND METHOD FOR MEASURING ANAEROBIC AMMONIUM OXIDATION ACTIVITY”, the entire content of which is incorporated herein by reference.

### TECHNICAL FIELD

The present disclosure belongs to the technical field of wastewater treatment, and particularly relates to a detection device and method for measuring anaerobic ammonium oxidation activity.

### BACKGROUND

Anaerobic ammonium oxidation is a biological reaction that uses ammonia nitrogen as an electron donor and nitrite as an electron acceptor. The anaerobic ammonium oxidation is first discovered in a fluidized bed reactor, and has been widely recognized as one of the most economically viable biological nitrogen removal processes due to its advantages such as no external addition of organic carbon sources, high nitrogen removal efficiency, low operating costs, and small footprint. In the wastewater treatment process, there are also other pathways such as a traditional nitrification-denitrification nitrogen removal pathway and a sulfur autotrophic nitrogen removal pathway, each playing significantly different roles in the nitrogen removal process. Therefore, how to distinguish the above pathways, especially the contribution rate of an anaerobic ammonium oxidation pathway, is an urgent problem to be solved in the process regulation and process engineering application procedure of anaerobic ammonium oxidation.

In the prior art, some use isotope-labeled NO₂ as a substrate to measure a production rate of labeled N₂ during the anaerobic ammonium oxidation process so as to calculate an anaerobic ammonium oxidation rate; and others calculate a total nitrogen removal rate by measuring changes in concentration of various nitrogen-containing components during processes such as denitrification and anaerobic ammonium oxidation in sludge, and then calculate an anaerobic ammonium oxidation process rate after excluding the denitrification process through difference values. In the prior art, the use of isotope tracing techniques or the collection of multiple water samples to measure various water quality indicators is required, resulting in heavy workloads, high costs, and complex equipment and instruments required.

### SUMMARY OF THE INVENTION

Therefore, the technical problem to be solved by the present disclosure is to overcome the defects in the prior art where NO₂⁻ is mostly used as a substrate to test an anaerobic ammonium oxidation rate, and this technique requires complex equipment, high monitoring costs, heavy workloads, numerous equipment and instruments used, and large human-induced errors. Thus, a detection device and method for measuring anaerobic ammonium oxidation activity are provided.

For this purpose, the present disclosure provides the following technical solutions.

The present disclosure provides a detection device for measuring anaerobic ammonium oxidation activity, including a reactor and a detector.

The reactor is configured to perform an anaerobic ammonium oxidation reaction and a denitrification reaction, wherein the reactor includes:
a first reaction chamber, configured to perform an anaerobic ammonium oxidation reaction and a denitrification reaction on sludge to be tested; and
a second reaction chamber, configured to perform a denitrification reaction on the sludge to be tested.

The detector is configured to control and record flow rates of reagents entering the first reaction chamber and the second reaction chamber, thereby ensuring that pH values in the first reaction chamber and the second reaction chamber are maintained at a constant and identical level.

The first reaction chamber and the second reaction chamber are communicable or separable via a separating plate.

Preferably, the first reaction chamber and the second reaction chamber have the same volume.

The detection device satisfies at least one of (1) to (2):
(1) a first sealing cover is provided on the top of the first reaction chamber; and
preferably, a first pH probe is provided on the sealing cover, and the detector monitors a pH value in the first reaction chamber through the first pH probe; or
(2) a second sealing cover is provided on the top of the second reaction chamber; and
preferably, a second pH probe is provided on the sealing cover, and the detector monitors a pH value in the second reaction chamber through the second pH probe.

The detection device satisfies at least one of (1) to (3):
(1) the first reaction chamber is further provided with a first gas dispersing unit for delivering gas to the first reaction chamber; and/or
a first reagent supply port, configured for supplying reagents; and
a first inlet, configured for introducing materials and/or discharging gas;
(2) the second reaction chamber is further provided with a second gas dispersing unit for delivering gas to the second reaction chamber; and/or
a second reagent supply port, configured for supplying reagents; and
a second inlet, configured for introducing materials and/or discharging gas; or
(3) the detection device is further provided with a constant temperature heater, configured for ensuring that the first reaction chamber and the second reaction chamber are maintained at a constant and identical temperature level.

The detector is further provided with a controller, and the controller is equipped with a built-in PID control algorithm and is configured to control and record the flow rates of the reagents entering the first reaction chamber and the second reaction chamber; and/or
the detector is further provided with a reagent storage tank.

The present disclosure further provides a detection method for measuring anaerobic ammonium oxidation activity, including:
(1) performing an anaerobic ammonium oxidation reaction and a denitrification reaction on sludge to be tested in a first reaction chamber, and performing a denitrification reaction on the sludge to be tested in a second reaction chamber, wherein the denitrification reactions in the first reaction chamber and the second reaction chamber have the same rate;
(2) controlling and recording, by a detector, flow rates of reagents entering the first reaction chamber and the second reaction chamber in a reaction process, thereby ensuring that pH in the first reaction chamber and pH in the second reaction chamber are maintained at a constant and identical level; wherein it is ensured that the denitrification reactions in the first reaction chamber and the second reaction chamber have the same rate by controlling the temperature and pH in the first reaction chamber and the second reaction chamber to be maintained at a constant and identical level; and
(3) calculating a real-time consumption rate of hydrogen ions based on concentrations of the reagents added to the first reaction chamber and the second reaction chamber, the flow rate of the reagent entering the first reaction chamber, and the flow rate of the reagent entering the second reaction chamber, and obtaining a real-time ammonia nitrogen conversion rate in an anaerobic ammonium oxidation process according to a correspondence relationship between hydrogen ion consumption rates and ammonia nitrogen consumption rates, namely real-time activity of anaerobic ammonium oxidation.

The detection method further includes: obtaining a maximum rate for anaerobic ammonium oxidation degradation of ammonia nitrogen based on the real-time activity of anaerobic ammonium oxidation.

Preferably, the detection method further includes: obtaining maximum specific activity of anaerobic ammonium oxidation activity based on the maximum rate for anaerobic ammonium oxidation degradation of ammonia nitrogen and a concentration of sludge.

Further, the real-time ammonia nitrogen conversion rate in the anaerobic ammonium oxidation process is calculated using Formula I;

$$\frac{d {C}_{NH}}{d t} = \frac{14 {C}_{H} ( {Q}_{A} - {Q}_{B} )}{0 \text{.13} V} \qquad Formula I$$

where dC_{NH}/dt is the real-time ammonia nitrogen conversion rate with a unit of mg·N·L⁻¹·min⁻¹;
t is time with a unit of min;
C_{H} is a hydrogen ion concentration in a total reagent supply amount of the first reaction chamber and the second reaction chamber with a unit of mmol/L, namely the real-time consumption rate of hydrogen ions; and the total reagent supply amount of the first reaction chamber and the second reaction chamber is calculated through the flow rates and concentrations of the reagents entering the first reaction chamber and the second reaction chamber;
Q^{A} is a flow rate of a reagent added to a chamber A with a unit of L/min;
Q^{B} is a flow rate of a reagent added to a chamber B with a unit of L/min;
0.13 is the number of hydrogen ions corresponding to 1 mol ammonia nitrogen consumed in the anaerobic ammonium oxidation reaction; and
V is a volume of a liquid phase in the first reaction chamber or the second reaction chamber with a unit of L.

Furthermore, maximum specific activity of anaerobic ammonium oxidation is calculated using Formula II:

$$μ = \frac{v}{M L V S S} \qquad Formula II$$

where v is the maximum rate for anaerobic ammonium oxidation degradation of ammonia nitrogen with a unit of mg·N·L⁻¹·min⁻¹;
MLVSS refers to a concentration of sludge in the first reaction chamber at the end of the reaction with a unit of mgMLVSS·L⁻¹; and
μ has a unit of mgN·min⁻¹·mgMLVSS⁻¹.

The detection method satisfies at least one of (1) to (6):
(1) during the anaerobic ammonium oxidation reaction and/or the denitrification reaction, a supplied reagent is a solution containing H⁺;
(2) a reagent supply amount is controlled by PID to ensure constant pH in the first reaction chamber and the second reaction chamber;
(3) a maximum rate v for anaerobic ammonium oxidation degradation of ammonia nitrogen is obtained based on a change curve of the real-time ammonia nitrogen conversion rate in the anaerobic ammonium oxidation process over time;
(4) nitrite and ammonia nitrogen are added into the first reaction chamber to perform the anaerobic ammonium oxidation reaction and the denitrification reaction;
preferably, a concentration of the nitrite in the first reaction chamber is not higher than 50 mg N/L; and
preferably, a concentration of the ammonia nitrogen in the first reaction chamber is not higher than 50 mg N/L;
(5) nitrite is added into the second reaction chamber to perform the denitrification reaction; and
preferably, a concentration of the nitrite in the second reaction chamber is not higher than 50 mg N/L; or
(6) a concentration of the sludge to be tested is 4000 mg/L to 6000 mg/L.

The technical solutions of the present disclosure have the following advantages.

1. The detection device for measuring the anaerobic ammonium oxidation activity provided by the present disclosure includes the reactor and the detector. The reactor performs the anaerobic ammonium oxidation reaction and the denitrification reaction, and includes the first reaction chamber and the second reaction chamber. The first reaction chamber is configured to perform the anaerobic ammonium oxidation reaction and the denitrification reaction on the sludge to be tested. The second reaction chamber is configured to perform the denitrification reaction on the sludge to be tested. The detector is configured to control and record the flow rates of the reagents entering the first reaction chamber and the second reaction chamber, thereby ensuring that the pH values in the first reaction chamber and the second reaction chamber are maintained at a constant and identical level. When the device is configured to measure the anaerobic ammonium oxidation activity, the real-time activity and the maximum activity of anaerobic ammonium oxidation can be obtained through the real-time consumption rate of the hydrogen ions in the reaction process, thereby avoiding the cumbersome process of continuously sampling and measuring the concentration of ammonia nitrogen or nitrite in a conventional method, simplifying the operation process, and reducing human-induced errors. The detection device can be system-integrated, and is convenient to carry due to the fact that no spectrophotometer, water quality analyzer or other instruments are required in the measuring process. The first reaction chamber and the second reaction chamber of the detection device use the same sludge as a control, and the interference caused by the denitrification process using endogenous organic matter as an electron donor is eliminated, improving the measurement precision.

2. In the detection device for measuring the anaerobic ammonium oxidation activity provided by the present disclosure, the detector in the detection device obtains the pH values in the reaction chambers through the pH probes, and controls the reagent supply amount, and thus equipment is simplified, costs are lowered, and the data acquisition frequency is high.

3. In the detection method for measuring the anaerobic ammonium oxidation activity provided by the present disclosure, when the anaerobic ammonium oxidation activity is measured through the detection method, the real-time activity and the maximum activity of anaerobic ammonium oxidation can be obtained through the consumption rate of the hydrogen ions in the reaction process, which replaces a method for measuring anaerobic ammonium oxidation activity by using ammonia nitrogen, nitrite and the like as substrates in traditional techniques. Compared with the traditional techniques, the detection method of the present disclosure shortens measurement time, reduces workloads and simplifies equipment. When the anaerobic ammonium oxidation activity is detected, the reactions are performed together in the first reaction chamber and the second reaction chamber, measurement errors caused by the denitrification process using the endogenous organic matter as the electron donor are eliminated, and the measurement precision is improved. At the same time, the constant pH value is further maintained, and the measuring precision for the H⁺ consumption amount is ensured.

In the detection method of the present disclosure, the reagent adding amount is controlled via PID, which maintains the constant pH and further ensures the measuring precision for the H⁺ consumption amount.

### BRIEF DESCRIPTION OF THE DRAWINGS

In order to describe the technical solutions in embodiments of the present disclosure or in the prior art more clearly, the accompanying drawings that need to be used in the descriptions for the embodiments or the prior art will be briefly introduced below. Apparently, the accompanying drawings in the following descriptions are some embodiments of the present disclosure, and for those of ordinary skill in the art, on the premise of no creative labor, other accompanying drawings can also be obtained from these accompanying drawings.

FIG. 1 is a detection device for measuring anaerobic ammonium oxidation activity in Example 1 of the present disclosure.

FIG. 2 is a real-time ammonia nitrogen conversion rate in an anaerobic ammonium oxidation process in Example 2 of the present disclosure.

### REFERENCE NUMERALS

1—First reaction chamber; 2—Second reaction chamber; 3—Gas storage tank; 4—First pH probe; 5—Second pH probe; 6—Controller; 7—Reagent storage tank; 8—Second reagent feed pump; 9—First reagent feed pump; 10—Separating plate;
1-1—First gas dispersing unit; 1-2—First stirrer; 1-3—First inlet; 1-4—First reagent supply port; 1-5—First sealing cover;
2-1—Second gas dispersing unit; 2-2—Second stirrer; 2-3—Second inlet; 2-4—Second reagent supply port; 2-5—Second sealing cover.

### DETAILED DESCRIPTION

The following examples are provided for a better understanding of the present disclosure and are not limited to the optimal embodiments, they do not limit the content and scope of protection of the present disclosure, and any product that is the same as or similar to the present disclosure obtained by anyone under the inspiration of the present disclosure or by combining the present disclosure with other features in the prior art falls within the scope of protection of the present disclosure.

If specific experimental steps or conditions are not specified in the examples, the operations or conditions of conventional experimental steps described in the literature in this art can be carried out. Adopted reagents or instruments which are not specified with manufacturers are conventional commercially-available reagent products.

### Example 1

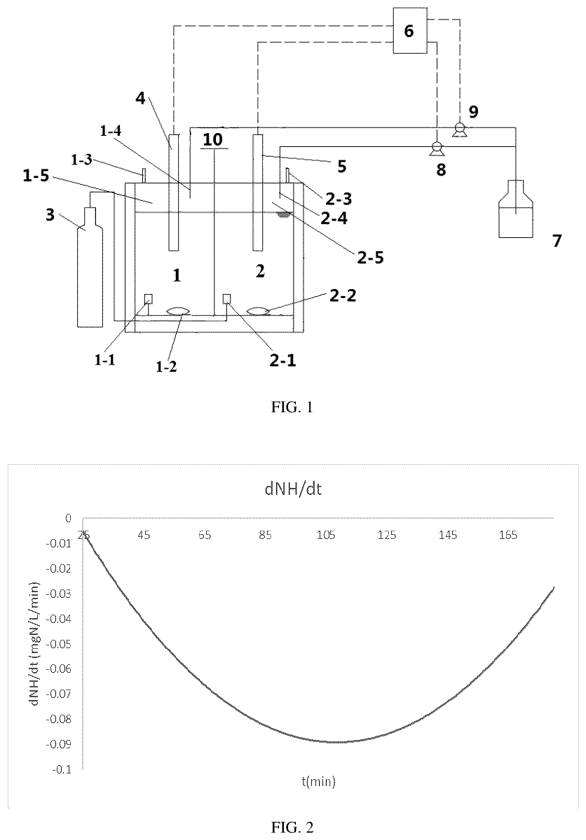

The present example provides a detection device for measuring anaerobic ammonium oxidation activity, as shown in FIG. 1, including a reactor and a detector.

The reactor is configured to perform an anaerobic ammonium oxidation reaction and a denitrification reaction. The reactor includes a first reaction chamber 1 and a second reaction chamber 2. The first reaction chamber 1 is configured to perform an anaerobic ammonium oxidation reaction and a denitrification reaction on sludge to be tested. The second reaction chamber 2 is configured to perform a denitrification reaction on the sludge to be tested. The reactor further includes a separating plate 10 which is able to move to achieve communication or separation between the first reaction chamber 1 and the second reaction chamber 2.

A first sealing cover 1-5 is provided on the top of the first reaction chamber 1 and configured to seal the first reaction chamber 1. A first gas dispersing unit 1-1 is provided on the bottom of the first reaction chamber 1 and configured to deliver gas in a gas storage tank 3 into the first reaction chamber 1. The first sealing cover of the first reaction chamber 1 is provided with a first inlet 1-3 and a first reagent supply port 1-4. The first inlet 1-3 is configured for discharging gas in the first reaction chamber 1 and/or delivering materials into the first reaction chamber. The first reagent supply port 1-4 is configured for supplying reagents.

A second sealing cover 2-5 is provided on the top of the second reaction chamber 2 and configured to seal the second reaction chamber 2. A second gas dispersing unit 2-1 is provided on the bottom of the second reaction chamber 2 and configured to deliver gas in the gas storage tank 3 into the second reaction chamber 2. The second sealing cover of the second reaction chamber 2 is provided with a second inlet 2-3 and a second reagent supply port 2-4. The second inlet 2-3 is configured for discharging gas in the second reaction chamber 2 and/or delivering materials into the second reaction chamber. The second reagent supply port 2-4 is configured for supplying reagents.

The detector is configured to control and record flow rates of reagents entering the first reaction chamber 1 and the second reaction chamber 2, thereby ensuring that pH values in the first reaction chamber 1 and the second reaction chamber 2 are maintained at a constant and identical level. The detector includes a first pH probe 4 and a second pH probe 5. The detector monitors a pH value in the first reaction chamber 1 through the first pH probe 4 and monitors a pH value in the second reaction chamber 2 through the second pH probe 5. The detector further includes a controller 6, a first reagent feed pump 9, a second reagent feed pump 8 and a reagent storage tank 7. The controller 6 is equipped with a built-in PID algorithm and is configured to control and record the flow rates of the reagents entering the first reaction chamber and the second reaction chamber. The reagents are supplied to the first reaction chamber 1 through the first reagent feed pump 9 and supplied to the second reaction chamber 2 through the second reagent feed pump 8. The reagents to be supplied are stored in the reagent storage tank 7.

Further, the temperatures in the first reaction chamber and the second reaction chamber are desired to be kept constant and identical. Preferably, the first reaction chamber and the second reaction chamber may be placed in a constant temperature heating device, such as a water bath heating device, to ensure the constant and identical temperature in the two reaction chambers. Furthermore, the temperatures in the first reaction chamber and the second reaction chamber may be obtained by temperature sensors, for example, a thermometer is arranged in the constant temperature heating device, and a temperature sensor may also be arranged inside the detector. There is no specific limitation on the above content.

Further, a first stirrer 1-2 is further arranged at the bottom of the first reaction chamber 1, a second stirrer 2-2 is arranged at the bottom of the second reaction chamber 2, and the stirrers are configured to stir materials in the reaction chambers. The stirrers may be, but are not limited to, stirring rotors.

In specific embodiments, the sludge to be tested, ammonia nitrogen and nitrite are subjected to the anaerobic ammonium oxidation reaction and the denitrification reaction in the first reaction chamber 1, and the sludge to be tested and the nitrite are subjected to the denitrification reaction in the second reaction chamber. The ammonia nitrogen may be, but is not limited to, ammonium sulfate and ammonia hydrochloride. The nitrite may be, but is not limited to, potassium nitrite and sodium nitrite. A concentration of the sludge to be tested is not specifically limited, and may be any concentration in a range from 4000 mg/L to 6000 mg/L. The sludge to be tested comes from an anaerobic ammonium oxidation reaction system under artificial or natural conditions. Use amount proportions of the sludge to be tested, the nitrite and the ammonia nitrogen are not specified.

Preferably, the first reaction chamber and the second reaction chamber have the same volume. Further, the use amounts of the sludge to be tested and the nitrite in the first reaction chamber and the second reaction chamber are the same, which ensures the same denitrification reaction rate.

Further, in specific embodiments, a hydrogen-ion containing solution is in the reagent storage tank 7, and may be, but is not limited to, a hydrochloric acid solution and a sulfuric acid solution. The gas in the gas storage tank 3 may be, but is not limited to, nitrogen.

### Example 2

The present example provides a detection method for measuring anaerobic ammonium oxidation activity. The detection method adopts the detection device provided in Example 1 and includes the following steps:

(1) an appropriate amount of anaerobic ammonium oxidation sludge is taken as sludge to be tested, rinsed with deionized water, and starved for 2 hours; a mixture of the sludge to be tested is placed into a reactor and diluted in proportion until the concentration of the sludge in the reactor is 4000 mg/L to 6000 mg/L, a first reaction chamber and a second reaction chamber are sealed, a separating plate is kept lifted to enable the first reaction chamber and the second reaction chamber to communicate with each other, stirrers 1-2 and 2-2 are turned on, nitrogen in a gas storage tank 3 is introduced into the first reaction chamber 1 and the second reaction chamber 2 through a first gas dispersing unit 1-1 and a second gas dispersing unit 2-1, after dissolved oxygen in the first reaction chamber and the second reaction chamber is exhausted, the introduction of the nitrogen is stopped, and the dissolved oxygen is discharged through a first inlet 1-3 and a second inlet 2-3; and after the dissolved oxygen is exhausted, the separating plate is pressed down to separate the first reaction chamber from the second reaction chamber. In the present example, the concentrations of the sludge to be tested diluted by water in the first reaction chamber and the second reaction chamber are both 4200 mg/L, wherein the sludge to be tested comes from an anaerobic ammonium oxidation reaction system under artificial or natural conditions.

Nitrite and ammonia nitrogen are added into the first reaction chamber 1 through the first inlet 1-3, and subjected to an anaerobic ammonium oxidation reaction and a denitrification reaction simultaneously in the first reaction chamber. Nitrite is added into the second reaction chamber 2 through the second inlet 2-3, the volume and concentration of the nitrite added into the first reaction chamber and the second reaction chamber are ensured to be the same, and the stirrers are kept rotating to ensure the same temperature and pH as well as the same denitrification reaction rate in the two chambers. In the present example, the nitrite in each reaction chamber is an aqueous NaNO₂ solution with a concentration of 400 mgN/L and an adding amount of 100 ml. The ammonia nitrogen in the first reaction chamber is an aqueous (NH₄)₂SO₄ solution with a concentration of 400 mgN/L and an adding amount of 100 ml. After the reagents are added, the volumes of the solutions in the two chambers are both constant as 1 L. There is no specific limitation on a mass ratio of the nitrite to the sludge to be tested. The first reaction chamber and the second reaction chamber are placed in a water bath heating device, a water bath temperature is controlled to be 30° C. to 35° C. and is kept constant, and the constant and identical temperature and pH may ensure the same denitrification reaction rate in the two reaction chambers.

(2) Reactions are initiated. In the reaction process, flow rates of reagents entering the first reaction chamber and the second reaction chamber are controlled and recorded by a detector to ensure that pH values in the two chambers remain constant and identical throughout the reactions. The detector is equipped with a built-in PID algorithm. Under PID control, rates of reagent feed pumps are adjusted according to the respective pH values in the two chambers and a system's preset target pH value of 7.5, so as to control the pH in both chambers at a constant level. Supplied reagents are added into the two reaction chambers through a first reagent supply port 1-4 and a second reagent supply port 2-4. Tuning of each PID parameter may be performed using a Cohen-Coon parameter tuning method or a Ziegler-Nichols parameter tuning method, and a control composition is as shown by the following formula:

$$u ( t ) = {{\overbrace{K ( e ( t )}}_{Proportional}}_{( P )} + {{\overbrace{\frac{1}{{T}_{i}} \int e ( t ) dt}}_{Integration}}_{( I )} + {{\overbrace{{T}_{d} \frac{d e ( t )}{d t} )}}_{Derivation}}_{( D )}$$

In this control, the proportional (P) part represents a difference between a control parameter and a target value, the integration (I) part represents an accumulated error, and the derivation (D) part represents prediction of future differences.

(3) According to a following stoichiometric equation of an anaerobic ammonium oxidation reaction process, hydrogen ions corresponding to the consumption of 1 mol of ammonia nitrogen in the anaerobic ammonium oxidation reaction may be obtained, that is, 0.13 mol of H⁺ is required for every 1 mol of ammonia nitrogen consumed.

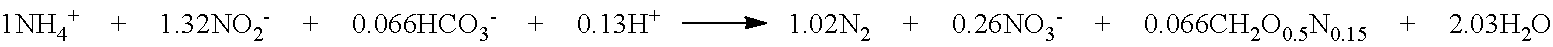

A real-time consumption rate of the hydrogen ions is calculated based on concentrations of the reagents added to the first reaction chamber and the second reaction chamber, the flow rate of the reagent entering the first reaction chamber, and the flow rate of the reagent entering the second reaction chamber, and a real-time ammonia nitrogen conversion rate in the anaerobic ammonium oxidation process is obtained according to a correspondence relationship between hydrogen ion consumption rates and ammonia nitrogen consumption rates, namely real-time activity of anaerobic ammonium oxidation. That is, the real-time ammonia nitrogen conversion rate in the anaerobic ammonium oxidation process is calculated using Formula I;

$$\frac{d {C}_{NH}}{d t} = \frac{14 {C}_{H} ( {Q}_{A} - {Q}_{B} )}{0 \text{.13} V} \qquad Formula I$$

where dC_{NH}/dt is the real-time ammonia nitrogen conversion rate with a unit of mgN·L⁻¹·min⁻¹;
t is time with a unit of min;
C_{H} is a hydrogen ion concentration in a total reagent supply amount of the first reaction chamber and the second reaction chamber, namely the real-time consumption rate of the hydrogen ions, with a unit of mmol/L; and the total reagent supply amount of the first reaction chamber and the second reaction chamber is calculated through the flow rates and concentrations of the reagents entering the first reaction chamber and the second reaction chamber;
Q^{A} is a flow rate of a reagent added to a chamber A with a unit of L/min;
Q^{B} is a flow rate of a reagent added to a chamber B with a unit of L/min;
0.13 is the number of hydrogen ions corresponding to 1 mol ammonia nitrogen consumed in the anaerobic ammonium oxidation reaction; and
V is a volume of a liquid phase in the first reaction chamber or the second reaction chamber with a unit of L. In the present example, the real-time ammonia nitrogen conversion rate in the anaerobic ammonium oxidation process is as shown in FIG. 2, namely the real-time activity of anaerobic ammonium oxidation.

(4) Further, a maximum rate v for anaerobic ammonium oxidation degradation of ammonia nitrogen is obtained based on a change curve of the real-time ammonia nitrogen conversion rate in the anaerobic ammonium oxidation process over time (i.e., dNH/dtₘₐₓ), with a unit of mgN·L⁻¹·min⁻¹.

Furthermore, maximum specific activity μ of anaerobic ammonium oxidation is calculated using Formula II

$$μ = \frac{v}{M L V S S} \qquad Formula II$$

where v is the maximum rate for anaerobic ammonium oxidation degradation of ammonia nitrogen with a unit of mgN/L/min;
MLVSS refers to a concentration of sludge in the first reaction chamber at the end of the reaction with a unit of mgMLVSS·L⁻¹; and
μ has a unit of mgN·min⁻¹·mgMLVSS⁻¹. In the present example, the concentration of the sludge in the first reaction chamber at the end of the reaction is 4200 mgMLVSS·L⁻¹, the maximum rate v for anaerobic ammonium oxidation degradation of ammonia nitrogen is 8.9×10⁻² mgN·L⁻¹·min⁻¹, and the maximum specific activity of anaerobic ammonium oxidation is 2.12×10⁻⁵ mgN·min⁻¹·mgMLVSS⁻¹.

In the detection method of the present example, when the anaerobic ammonium oxidation activity is measured, the anaerobic ammonium oxidation activity can be obtained through the consumption rate of the hydrogen ions in the reaction process, which replaces a method for measuring anaerobic ammonium oxidation activity by using ammonia nitrogen, nitrite and the like as substrates in traditional techniques. Compared with the traditional techniques, the detection method of the present disclosure shortens measurement time, reduces workloads and simplifies equipment. When the anaerobic ammonium oxidation activity is detected, the reactions are performed together in the first reaction chamber and the second reaction chamber, measurement errors caused by the denitrification process using endogenous organic matter as an electron donor are eliminated, and the measurement precision is improved.

Obviously, the above examples are only instances for clear explanation, rather than limiting the embodiments. For those of ordinary skill in the art, other different forms of changes or variations can further be made based on the above explanation. It is not necessary and impossible to exhaustively list all embodiments here. The obvious changes or variations arising from this are still within the scope of protection of the present disclosure.

1. A detection device for measuring anaerobic ammonium oxidation activity, comprising a reactor and a detector, wherein
the reactor is configured to perform an anaerobic ammonium oxidation reaction and a denitrification reaction, wherein the reactor comprises:
a first reaction chamber, configured to perform an anaerobic ammonium oxidation reaction and a denitrification reaction on sludge to be tested; and
a second reaction chamber, configured to perform a denitrification reaction on the sludge to be tested; and
the detector is configured to control and record flow rates of reagents entering the first reaction chamber and the second reaction chamber, thereby ensuring that pH values in the first reaction chamber and the second reaction chamber are maintained at a constant and identical level.

2. The detection device according to claim 1, wherein the first reaction chamber and the second reaction chamber are communicable or separable via a separating plate; and
preferably, the first reaction chamber and the second reaction chamber have the same volume.

3. The detection device according to claim 1, wherein at least one of (1) to (2) is satisfied:
(1) a first sealing cover is provided on the top of the first reaction chamber; and
preferably, a first pH probe is provided on the sealing cover, and the detector monitors a pH value in the first reaction chamber through the first pH probe; or
(2) a second sealing cover is provided on the top of the second reaction chamber; and
preferably, a second pH probe is provided on the sealing cover, and the detector monitors a pH value in the second reaction chamber through the second pH probe.

4. The detection device according to claim 1, wherein at least one of (1) to (3) is satisfied:
(1) the first reaction chamber is further provided with a first gas dispersing unit for delivering gas to the first reaction chamber; and/or
a first reagent supply port, configured for supplying reagents; and
a first inlet, configured for introducing materials and/or discharging gas;
(2) the second reaction chamber is further provided with a second gas dispersing unit for delivering gas to the second reaction chamber; and/or
a second reagent supply port, configured for supplying reagents; and
a second inlet, configured for introducing materials and/or discharging gas; or
(3) the detection device is further provided with a constant temperature heater, configured for ensuring that the first reaction chamber and the second reaction chamber are maintained at a constant and identical temperature level.

5. The detection device according to claim 1, wherein the detector is further provided with a controller, and the controller is equipped with a built-in PID control algorithm and is configured to control and record the flow rates of the reagents entering the first reaction chamber and the second reaction chamber; and/or
the detector is further provided with a reagent storage tank.

6. A detection method for measuring anaerobic ammonium oxidation activity, comprising:
(1) performing an anaerobic ammonium oxidation reaction and a denitrification reaction on sludge to be tested in a first reaction chamber, and performing a denitrification reaction on the sludge to be tested in a second reaction chamber, wherein the denitrification reactions in the first reaction chamber and the second reaction chamber have the same rate;
(2) controlling and recording, by a detector, flow rates of reagents entering the first reaction chamber and the second reaction chamber in a reaction process, ensuring that pH in the first reaction chamber and pH in the second reaction chamber are maintained at a constant and identical level; and
(3) calculating a real-time consumption rate of hydrogen ions based on concentrations of the reagents added to the first reaction chamber and the second reaction chamber, the flow rate of the reagent entering the first reaction chamber, and the flow rate of the reagent entering the second reaction chamber, and obtaining a real-time ammonia nitrogen conversion rate in an anaerobic ammonium oxidation process according to a correspondence relationship between hydrogen ion consumption rates and ammonia nitrogen consumption rates, namely real-time activity of anaerobic ammonium oxidation.

7. The detection method according to claim 6, further comprising: obtaining a maximum rate for anaerobic ammonium oxidation degradation of ammonia nitrogen based on the real-time activity of anaerobic ammonium oxidation; and
preferably, further comprising: obtaining maximum specific activity of anaerobic ammonium oxidation activity based on the maximum rate for anaerobic ammonium oxidation degradation of ammonia nitrogen and a concentration of sludge.

8. The detection method according to claim 6, wherein the real-time ammonia nitrogen conversion rate in the anaerobic ammonium oxidation process is calculated using Formula I;
$$\frac{d {C}_{NH}}{d t} = \frac{14 {C}_{H} ( {Q}_{A} - {Q}_{B} )}{0 \text{.13} V} \qquad Formula I$$
where dC_{NH}/dt is the real-time ammonia nitrogen conversion rate with a unit of mgN·L⁻¹·min⁻¹;
t is time with a unit of min;
C_{H} is a hydrogen ion concentration in a total reagent supply amount of the first reaction chamber and the second reaction chamber with a unit of mmol/L;
Q_{A} is a flow rate of a reagent added to a chamber A with a unit of L/min;
Q_{B} is a flow rate of a reagent added to a chamber B with a unit of L/min;
0.13 is the number of hydrogen ions corresponding to 1 mol ammonia nitrogen consumed in the anaerobic ammonium oxidation reaction; and
V is a volume of a liquid phase in the first reaction chamber or the second reaction chamber with a unit of L.

9. The detection method according to claim 7, wherein the maximum specific activity μ of anaerobic ammonium oxidation is calculated using Formula II:
$$μ = \frac{v}{M L V S S} \qquad Formula II$$
where v is the maximum rate for anaerobic ammonium oxidation degradation of ammonia nitrogen with a unit of mgN·L⁻¹·min⁻¹;
MLVSS refers to a concentration of sludge in the first reaction chamber at the end of the reaction with a unit of mgMLVSS·L⁻¹; and
μ has a unit of mgN·min⁻¹·mgMLVSS⁻¹.

10. The detection method according to claim 6, wherein at least one of (1) to (6) is satisfied:
(1) during the anaerobic ammonium oxidation reaction and/or the denitrification reaction, a supplied reagent is a solution containing H⁺;
(2) a reagent supply amount is controlled by PID to ensure constant pH in the first reaction chamber and the second reaction chamber;
(3) a maximum rate v for anaerobic ammonium oxidation degradation of ammonia nitrogen is obtained based on a change curve of the real-time ammonia nitrogen conversion rate in the anaerobic ammonium oxidation process over time;
(4) nitrite and ammonia nitrogen are added into the first reaction chamber to perform the anaerobic ammonium oxidation reaction and the denitrification reaction;
preferably, a concentration of the nitrite in the first reaction chamber is not higher than 50 mg N/L; and
preferably, a concentration of the ammonia nitrogen in the first reaction chamber is not higher than 50 mg N/L;
(5) nitrite is added into the second reaction chamber to perform the denitrification reaction; and
preferably, a concentration of the nitrite in the second reaction chamber is not higher than 50 mg N/L; or
(6) a concentration of the sludge to be tested is 4000 mg/L to 6000 mg/L.

11. The detection device according to claim 2, wherein at least one of (1) to (2) is satisfied:
(1) a first sealing cover is provided on the top of the first reaction chamber; and
preferably, a first pH probe is provided on the sealing cover, and the detector monitors a pH value in the first reaction chamber through the first pH probe; or
(2) a second sealing cover is provided on the top of the second reaction chamber; and
preferably, a second pH probe is provided on the sealing cover, and the detector monitors a pH value in the second reaction chamber through the second pH probe.

12. The detection device according to claim 2, wherein at least one of (1) to (3) is satisfied:
(1) the first reaction chamber is further provided with a first gas dispersing unit for delivering gas to the first reaction chamber; and/or
a first reagent supply port, configured for supplying reagents; and
a first inlet, configured for introducing materials and/or discharging gas;
(2) the second reaction chamber is further provided with a second gas dispersing unit for delivering gas to the second reaction chamber; and/or
a second reagent supply port, configured for supplying reagents; and
a second inlet, configured for introducing materials and/or discharging gas; or
(3) the detection device is further provided with a constant temperature heater, configured for ensuring that the first reaction chamber and the second reaction chamber are maintained at a constant and identical temperature level.

13. The detection device according to claim 3, wherein at least one of (1) to (3) is satisfied:
(1) the first reaction chamber is further provided with a first gas dispersing unit for delivering gas to the first reaction chamber; and/or
a first reagent supply port, configured for supplying reagents; and
a first inlet, configured for introducing materials and/or discharging gas;
(2) the second reaction chamber is further provided with a second gas dispersing unit for delivering gas to the second reaction chamber; and/or
a second reagent supply port, configured for supplying reagents; and
a second inlet, configured for introducing materials and/or discharging gas; or
(3) the detection device is further provided with a constant temperature heater, configured for ensuring that the first reaction chamber and the second reaction chamber are maintained at a constant and identical temperature level.

14. The detection device according to claim 3, wherein the detector is further provided with a controller, and the controller is equipped with a built-in PID control algorithm and is configured to control and record the flow rates of the reagents entering the first reaction chamber and the second reaction chamber; and/or
the detector is further provided with a reagent storage tank.

15. The detection method according to claim 7, wherein at least one of (1) to (6) is satisfied:
(1) during the anaerobic ammonium oxidation reaction and/or the denitrification reaction, a supplied reagent is a solution containing H⁺;
(2) a reagent supply amount is controlled by PID to ensure constant pH in the first reaction chamber and the second reaction chamber;
(3) a maximum rate v for anaerobic ammonium oxidation degradation of ammonia nitrogen is obtained based on a change curve of the real-time ammonia nitrogen conversion rate in the anaerobic ammonium oxidation process over time;
(4) nitrite and ammonia nitrogen are added into the first reaction chamber to perform the anaerobic ammonium oxidation reaction and the denitrification reaction;
preferably, a concentration of the nitrite in the first reaction chamber is not higher than 50 mg N/L; and
preferably, a concentration of the ammonia nitrogen in the first reaction chamber is not higher than 50 mg N/L;
(5) nitrite is added into the second reaction chamber to perform the denitrification reaction; and
preferably, a concentration of the nitrite in the second reaction chamber is not higher than 50 mg N/L; or
(6) a concentration of the sludge to be tested is 4000 mg/L to 6000 mg/L.

16. The detection method according to claim 8, wherein at least one of (1) to (6) is satisfied:
(1) during the anaerobic ammonium oxidation reaction and/or the denitrification reaction, a supplied reagent is a solution containing H⁺;
(2) a reagent supply amount is controlled by PID to ensure constant pH in the first reaction chamber and the second reaction chamber;
(3) a maximum rate v for anaerobic ammonium oxidation degradation of ammonia nitrogen is obtained based on a change curve of the real-time ammonia nitrogen conversion rate in the anaerobic ammonium oxidation process over time;
(4) nitrite and ammonia nitrogen are added into the first reaction chamber to perform the anaerobic ammonium oxidation reaction and the denitrification reaction;
preferably, a concentration of the nitrite in the first reaction chamber is not higher than 50 mg N/L; and
preferably, a concentration of the ammonia nitrogen in the first reaction chamber is not higher than 50 mg N/L;
(5) nitrite is added into the second reaction chamber to perform the denitrification reaction; and
preferably, a concentration of the nitrite in the second reaction chamber is not higher than 50 mg N/L; or
(6) a concentration of the sludge to be tested is 4000 mg/L to 6000 mg/L.

17. The detection method according to claim 9, wherein at least one of (1) to (6) is satisfied:
(1) during the anaerobic ammonium oxidation reaction and/or the denitrification reaction, a supplied reagent is a solution containing H⁺;
(2) a reagent supply amount is controlled by PID to ensure constant pH in the first reaction chamber and the second reaction chamber;
(3) a maximum rate v for anaerobic ammonium oxidation degradation of ammonia nitrogen is obtained based on a change curve of the real-time ammonia nitrogen conversion rate in the anaerobic ammonium oxidation process over time;
(4) nitrite and ammonia nitrogen are added into the first reaction chamber to perform the anaerobic ammonium oxidation reaction and the denitrification reaction;
preferably, a concentration of the nitrite in the first reaction chamber is not higher than 50 mg N/L; and
preferably, a concentration of the ammonia nitrogen in the first reaction chamber is not higher than 50 mg N/L;
(5) nitrite is added into the second reaction chamber to perform the denitrification reaction; and
preferably, a concentration of the nitrite in the second reaction chamber is not higher than 50 mg N/L; or
(6) a concentration of the sludge to be tested is 4000 mg/L to 6000 mg/L.
